(19) United States
(12) Patent Application Publication
(10) Pub. No.: US 2005/0100606 A1
(43) Pub. Date: May 12, 2005

(54) Controlled release formulation of tamsulosin hydrochloride and preparation process thereof

(75) Inventors: Hun Sik Wang, Seongnam-city (KR); Jun Sang Park, Seongnam-city (KR); Min Chang Kwon, Seoul (KR); Ji Yeon Shim, Seongnam-city (KR); Bong Sang Lee, Suwon-city (KR); Hong Ryeol Jeon, Suwon-city (KR)

Correspondence Address: LEYDIG VOIT & MAYER, LTD, 700 THIRTEENTH ST. NW, SUITE 300, WASHINGTON, DC, 20005-3960, US

(73) Assignees: GL PharmTech Corp., Seongnam-city (KR); CTC BIO, INC., Seoul (KR)

(21) Appl. No.: 10/747,044

(22) Filed: Dec. 30, 2003

(30) Foreign Application Priority Data
Nov. 7, 2003 (KR) 2003-78647

(51) Int. Cl. A61K 9/26

(52) U.S. Cl. 424/470

(57) ABSTRACT

Provided is a controlled-release formulation of tamsulosin hydrochloride, which includes a granular core, and a drug-coating layer coated on the granular core, including the tamsulosin hydrochloride and a release-controlling agent selected from the group consisting of (a) a first copolymer of 1 part by weight of ethylacrylate, 2 parts by weight of methylmethacrylate, and 0.1 parts by weight of trimethylammonioethyl methacrylate chloride, (b) a mixture of the first copolymer and a second copolymer of 1 part by weight of ethylacrylate, 2 parts by weight of methylmethacrylate, and 0.2 parts by weight of trimethylammonioethyl methacrylate chloride, the weight ratio between the first copolymer and the second copolymer being 1: 0.05 to 0.2, and (c) polyvinylacetate. A process for preparing the controlled-release formulation of the tamsulosin hydrochloride is also provided.

### BACKGROUND OF THE INVENTION

This application claims priority from Korean Patent Application No. 2003-78647, filed on Nov. 7, 2003, in the Korean Intellectual Property Office, the disclosure of which is incorporated herein by reference in its entirety.

1. Field of the Invention

The present invention relates to a controlled release formulation of tamsulosin hydrochloride, and more particularly, to a controlled release formulation of tamsulosin hydrochloride and a preparation process thereof.

2. Description of the Related Art

Tamsulosin hydrochloride is an alpha (α)1 blocker that is specific to prostatic smooth muscle. Tamsulosin hydrochloride is a drug useful for the treatment of obstructive urinary tract disorders due to benign prostate enlargement. The usual daily dose of tamsulosin hydrochloride is 0.2 to 0.8 mg. Tamsulosin hydrochloride has greater selectivity for alpha1 in prostatic smooth muscle and acts weakly on blood vessels, unlike other alpha1 blockers such as doxazosin and terazosin, thereby greatly decreasing occurrences of side effects such as orthostatic hypotension. However, despite the high selectivity for alpha1 in prostatic smooth muscle, since tamsulosin hydrochloride is highly potent, a very low dose is required. Also, excessive increase of the initial level of tamsulosin hydrochloride in the blood due to fast absorption into the body may cause side effects such as orthostatic hypotension.

In view of these problems, U.S. Pat. No. 4,772,475 discloses a controlled release formulation of tamsulosin hydrochloride. According to this patent, tamsulosin hydrochloride is mixed with at least 50% by weight of a unit-forming substance selected from the group consisting of crystalline cellulose, chitin, and chitosan. Then, to the mixture, there is added a release-controlling agent selected from the group consisting of aqueous suspension, aqueous emulsion, aqueous gel, and water-containing organic solvent solution of water-insoluble polymers such as acrylic acid based polymers, acrylic acid based copolymers, and cellulose derivatives, and water itself, followed by granulation, to produce controlled-release granules containing tamsulosin hydrochloride. As disclosed in the above patent, the unit-forming substance is preferably crystalline or microcrystalline cellulose. Also, the release-controlling agent is preferably an aqueous suspension of methacrylic acid-ethylacrylate copolymer (trade name: Eudragit L100-55), an aqueous suspension of ethyl cellulose, or water itself.

However, the controlled-release formulation according to the above patent has poor controlled-release. For example, at one hour after start of release in a first acidic solution (pH: about 1.2), 16.2 to 60.4% of a drug is released. When release is carried out in a second neutral solution (pH: about 6.8) after one hour of the start of release in the first acid solution, more than 90% of the drug is released within one hour after start of release in the second neutral solution.

The methacrylic acid-ethylacrylate copolymer (trade name: Eudragit L100-55) used as a release-controlling agent in the above patent forms a type of a matrix structure to control the release of a drug. The methacrylic acid-ethylacrylate copolymer has been known to be used for enteric coating and exhibits a pH-dependent solubility. That is, the methacrylic acid-ethylacrylate copolymer (trade name: Eudragit L100-55) is not dissolved at less than about pH 5.5. However, at more than pH 5.5, its solubility greatly increases. In this regard, a matrix structure made of the above copolymer is rapidly dissolved at more than pH 5.5, thereby increasing the release of a drug. This property can be developed into a more serious problem in that the pH of the intestinal tract in the human body can vary within the range of 4 to 8 depending on food intake, stress, and illness condition of patients [Gastric Retentive Drug-Delivery System, S J Hwang et al, Critical Reviews in Therapeutic Drug Carrier Systems, 15(3), p. 243, 1988; Influence of Food on the Bioavailability of Drugs, A Melander, Clinical Pharmacokinetics, 3, p. 337, 1978]. Meanwhile, it is known that the mobility of the gastric intestinal tract is greatly changed depending on the condition of a patient. Such mobility change of the gastric intestinal tract can also act as a factor that influences the release rate of a drug, together with the ambient pH.

Products containing tamsulosin hydrochloride commercially available (Harnal, Yamanouchi Co. Ltd.; Flomax, Boehringer Ingelheim) also exhibit distinct pH-dependent drug release property. It is reported that administration of these products into the human body before food intake results in 30% increase in bioavailability of these products and 40 to 70% increase in maximum drug concentration in bloods (Cmax), as compared to after food intake [Physicians Desk Reference 2002]. This report demonstrates that food intake influences the mobility of the gastric intestinal tract and the ambient pH of a drug-containing formulation. Furthermore, high drug concentration in bloods before food intake may easily cause the above-described side effects. In this regard, the administration of the above products after food intake is recommended. In such case, however, there is a problem in that the bioavailability of the drug is lowered.

Therefore, in order to solve the disadvantages of the above-described technologies and commercially available products, there is a need to design a tamsulosin hydrochloride-containing formulation so that the release behavior of the tamsulosin hydrochloride from the formulation is none at all or slightly influenced by the ambient pH in vivo.

Meanwhile, U.S. Pat. No. 6,328,979 discloses a sustained-release composition which includes an ionic drug such as tamsulosin hydrochloride and an ionic substance having an opposite charge to that of the ionic drug for increasing hydrophobicity of the ionic drug. However, the sustained-release composition is used only for injectable formulation.

### SUMMARY OF THE INVENTION

The present invention provides a controlled-release formulation. The controlled-release formulation maintains similar drug release profiles irrespective of the ambient pH. Therefore, side effects such as orthostatic hypotension and abnormal release can be reduced upon the administration of tamsulosin hydrochloride. Furthermore, the effectiveness of a drug can be maintained longer. The present invention also provides a simplified process for preparing a controlled-release formulation.

According to an aspect of the present invention, there is provided a controlled-release formulation of tamsulosin hydrochloride, which comprises: a granular core; and a drug-coating layer coated on the granular core, comprising the tamsulosin hydrochloride and a release-controlling agent selected from the group consisting of (a) a first copolymer of 1 part by weight of ethylacrylate, 2 parts by weight of methylmethacrylate, and 0.1 parts by weight of trimethylammonioethyl methacrylate chloride, (b) a mixture of the first copolymer and a second copolymer of 1 part by weight of ethylacrylate, 2 parts by weight of methylmethacrylate, and 0.2 parts by weight of trimethylammonioethyl methacrylate chloride, the weight ratio between the first copolymer and the second copolymer being 1: 0.05 to 0.2, and (c) polyvinylacetate.

According to another aspect of the present invention, there is provided a process for preparing a controlled-release formulation of tamsulosin hydrochloride, which comprises: preparing a drug-coating solution comprising the tamsulosin hydrochloride and a release-controlling agent selected from the group consisting of (a) a first copolymer of 1 part by weight of ethylacrylate, 2 parts by weight of methylmethacrylate, and 0.1 parts by weight of trimethylammonioethyl methacrylate chloride, (b) a mixture of the first copolymer and a second copolymer of 1 part by weight of ethylacrylate, 2 parts by weight of methylmethacrylate, and 0.2 parts by weight of trimethylammonioethyl methacrylate chloride, the weight ratio between the first copolymer and the second copolymer being 1: 0.05 to 0.2, and (c) polyvinylacetate; and spray-coating a granular core with the drug-coating solution to form a drug-coating layer.

### BRIEF DESCRIPTION OF THE DRAWING

The above and other features and advantages of the present invention will become more apparent by describing in detail exemplary embodiments thereof with reference to the attached drawing in which:

FIG. 1 is a view showing the results of release rate tests for controlled-release formulations according to the present invention.

### DETAILED DESCRIPTION OF THE INVENTION

The present invention provides a controlled-release formulation of tamsulosin hydrochloride, which includes: a granular core; and a drug-coating layer coated on the granular core, including the tamsulosin hydrochloride and a release-controlling agent selected from the group consisting of (a) a first copolymer of 1 part by weight of ethylacrylate, 2 parts by weight of methylmethacrylate, and 0.1 parts by weight of trimethylammonioethyl methacrylate chloride, (b) a mixture of the first copolymer and a second copolymer of 1 part by weight of ethylacrylate, 2 parts by weight of methylmethacrylate, and 0.2 parts by weight of trimethylammonioethyl methacrylate chloride, the weight ratio between the first copolymer and the second copolymer being 1: 0.05 to 0.2, and (c) polyvinylacetate.

The controlled-release formulation of tamsulosin hydrochloride according to the present invention includes the granular core and the drug-coating layer that includes the release-controlling agent and the tamsulosin hydrochloride. Preferably, the controlled-release formulation further includes a release-controlling layer coated on the drug-coating agent with the release-controlling agent. The release-controlling layer serves to still more retard the release rate of the tamsulosin hydrochloride from the controlled-release formulation and to suspend the release of the tamsulosin hydrochloride for a predetermined duration.

As used herein, the granular core may be a water-soluble or -insoluble granular core commonly used in granulation. For example, a sugar sphere such as Non-Pareil, Nu-Core, and Nu-Pareil may be used. A sugar sphere mixed with corn starch may also be used.

The release-controlling agent may be selected from the group consisting of the first copolymer of 1 part by weight of ethylacrylate, 2 parts by weight of methylmethacrylate, and 0.1 parts by weight of trimethylammonioethyl methacrylate chloride, the mixture (1: 0.05 to 2, by weight) of the first copolymer and the second copolymer of 1 part by weight of ethylacrylate, 2 parts by weight of methylmethacrylate, and 0.2 parts by weight of trimethylammonioethyl methacrylate chloride, and the polyvinylacetate.

Preferably, the first copolymer is Eudragit RS (Degussa) and the second copolymer is Eudragit RL (Degussa). Preferably, the polyvinylaceate is commercially available Kollicoat SR (BASF Co.).

There are known Eudragit RS and RL of various phases such as liquid, suspension, and powder. Examples of the Eudragit RS and RL include Eudragit RS 12.5P (organic solution), RS 100 (granule), RS PO (powder), RS 30D (aqueous dispersion), RL 12.5P (organic solution), RL 100 (granule), RL PO (powder), and RL 30D (aqueous dispersion). The Eudragit RS or a combination of the Eudragit RS and RL of the above-listed various phases may be used as the release-controlling agent of the present invention. In particular, preferred are the Eudragit RS 30D and a combination of the Eudragit RS 30D and RL 30D in the form of an aqueous dispersion that require no separate organic solvents and minimizes environmental pollution.

The release-controlling agent forms a matrix-type, drug-coating layer on the granular core, together with the tamsulosin hydrochloride. The controlled-release formulation of the present invention thus prepared serves to efficiently prevent the rapid penetration of a body fluid on contact with the body fluid and the rapid diffusion of the tamsulosin hydrochloride toward the outside of the matrix. Furthermore, the controlled-release formulation exhibits constant release property regardless of the ambient pH of the formulation.

A weight ratio between the tamsulosin hydrochloride and the release-controlling agent in the drug-coating layer may be 1:10 to 1:200, preferably 1:30 to 1:150.

The release-controlling agent in the release-controlling layer may be used in an amount of 20 to 80 parts by weight, preferably 30 to 50 parts by weight, based on 1 part by weight of the tamsulosin hydrochloride.

The drug-coating layer and/or the release-controlling layer may further include 0.1 to 5 parts by weight, preferably 0.5 to 3 parts by weight of a release-controlling adjuvant selected from the group consisting of polyvinylpyrrolidone (also called povidone), hydroxypropylmethyl cellulose, hydroxypropyl cellulose, and a mixture thereof, based on 1 part by weight of the tamsulosin hydrochloride. The release-controlling adjuvant is dissolved upon contact with a medium after administration and then forms a channel in the drug-coating layer and/or the release-controlling layer. The channel can act as a path for diffusion of the tamsulosin hydrochloride. Therefore, when the release of the tamsulosin hydrochloride is too slow, the release-controlling adjuvant can facilitate the release rate of the tamsulosin hydrochloride.

Preferably, the drug-coating layer and/or the release-controlling layer further include(s) a plasticizer selected from the group consisting of dibutylsebacate, triethylcitrate, propylene glycol, and a mixture thereof. Plasticizers are generally used to facilitate polymer coating film formation, but can change the release rate of the tamsulosin hydrochloride by varying the types and amounts of the plasticizers. Therefore, the plasticizer as used herein acts as a release-controlling adjuvant, as well as effects intrinsic functions. The plasticizer that can be used herein is preferably dibutylsebacate, triethylcitrate, propylene glycol, or a mixture thereof. The content of the plasticizer may vary depending on a desired drug release pattern and the type of the plasticizer.

The controlled-release formulation of the present invention may further include a diluent that is commonly used in the pharmaceutical industry, for example, an opaquant such as titanium oxide, a lubricant such as talc and magnesium stearate, and a colorant.

As seen from Examples as will be described later, the controlled-release formulation of the present invention exhibits the pH-independent release property of the tamsulosin hydrochloride. Therefore, a unique drug release rate even when pH environments of the gastric intestinal tract are changed after administration can be ensured.

The controlled-release formulation of the present invention may further include an enteric coating layer when needed. The enteric coating layer serves to suspend the drug release in the gastric tract and to facilitate the drug release after delivery of the formulation into the intestinal tract.

The present invention also provides a process for preparing a controlled-release formulation of tamsulosine hydrochloride. In detail, the process includes preparing a drug-coating solution that includes the tamsulosin hydrochloride and a release-controlling agent selected from the group consisting of (a) a first copolymer of 1 part by weight of ethylacrylate, 2 parts by weight of methylmethacrylate, and 0.1 parts by weight of trimethylammonioethyl methacrylate chloride, (b) a mixture (1: 0.05 to 2, by weight) of the first copolymer and a second copolymer of 1 part by weight of ethylacrylate, 2 parts by weight of methylmethacrylate, and 0.2 parts by weight of trimethylammonioethyl methacrylate chloride, and (c) polyvinylacetate; and spray-coating a granular core with the drug-coating solution to form a drug-coating layer.

The process may further include coating the drug-coating layer with the release-controlling agent to form a release-controlling layer.

The granular core is as described above. A weight ratio between the tamsulosin hydrochloride and the release-controlling agent in the drug-coating layer may be 1:10 to 1:200, preferably 1:30 to 1:150. The release-controlling agent in the release-controlling agent may be used in an amount of 20 to 80 parts by weight, preferably 30 to 50 parts by weight, based on 1 part by weight of the tamsulosin hydrochloride.

The drug-coating layer and/or the release-controlling layer may further include 0.1 to 5 parts by weight, preferably 0.5 to 3 parts by weight of a release-controlling adjuvant selected from the group consisting of polyvinylpyrrolidone, hydroxypropylmethyl cellulose, hydroxypropyl cellulose, and a mixture thereof, based on 1 part by weight of the tamsulosin hydrochloride. The drug-coating layer and/or the release-controlling layer may further include a plasticizer selected from the group consisting of dibutylsebacate, triethylcitrate, propylene glycol, and a mixture thereof. The release-controlling adjuvant and the plasticizer may be added to a coating solution for the drug-coating layer and/or the release-controlling layer and then mixed.

The coating solution may include an aqueous dispersion of Eudragit RS 30D, a combination of Eudragit RS 30D and Eudragit RL 30D, or polyvinylacetate as the release-controlling agent. Therefore, there is no need to use a separate solvent for dissolving the release-controlling agent and the drug (tamsulosin hydrochloride), thereby simplifying a preparation process. When solid Eudragit RS or RL is used, water, ethanol, acetone, dichloromethane, or a mixture thereof may be used.

The controlled-release formulation of the present invention may further include a diluent that is commonly used in the pharmaceutical industry, for example, an opaquant such as titanium oxide, a lubricant such as talc and magnesium stearate, and a colorant.

The process of the present invention may further include coating the drug-coating layer or the release-controlling layer with an enteric coating agent selected from the group consisting of carboxymethylethyl cellulose, hydroxypropylmethyl cellulose phthalate, hydroxypropylmethyl cellulose acetate succinate, cellulose acetate phthalate, a copolymer of methacryic acid and ethylacrylate, a copolymer of methacrylic acid and methylmethacrylate, and shellac to form an enteric coating layer.

The spray-coating may be carried out using a conventional fluidized bed system with bottom spray, a centrifugal granulator or coater, SPIR-A-FLOW (Freund, Japan), or Granurex (Freund, Japan).

The formation of the individual layers, i.e., the drug-coating layer, the release-controlling layer, and the enteric coating layer may be carried out in situ. A subsequent drying process may be also carried out in situ. Therefore, the process of the present invention is simplified and a manufacturing cost is reduced, relative to conventional processes in which granulation and drying are separately carried out (U.S. Pat. No. 4,772,475; Korean Patent No. 67,674). Furthermore, a unit working capacity can be easily increased, thereby increasing productivity.

The controlled-release formulation of the present invention may be in various pharmaceutical forms. Preferably, the controlled-release formulation is in the form of granules. Also, the controlled-release formulation may be made into granules and then filled into appropriate capsules or may be made into tablets.

Hereinafter, the present invention will be described more specifically by Examples. However, the following Examples are provided only for illustrations and thus the present invention is not limited to or by them.

### EXAMPLE 1

1.078 g of tamsulosin hydrochloride, 87.6 g of talc, and 13.14 g of propylene glycol were added to 438 g of Eudragit RS 30D (Degussa) and completely mixed to prepare a coating solution. The coating solution was gradually added to a Granurex GX20 (Freund, Japan) containing about 700 g of a sugar sphere at outlet temperature of about 40° C. to prepare granules.

### EXAMPLE 2

1.078 g of tamsulosin hydrochloride, 43.8 g of talc, and 13.14 g of triethylcitrate were added to 219 g of Eudragit RS 30D (Degussa) and completely mixed to prepare a coating solution. The coating solution was gradually added to a Granurex GX20 (Freund, Japan) containing about 700 g of a sugar sphere at outlet temperature of about 40° C. to prepare granules.

### EXAMPLE 3

1.078 g of tamsulosin hydrochloride, 43.8 g of talc, and 13.14 g of dibutylsebacate were added to 219 g of Eudragit RS 30D (Degussa) and completely mixed to prepare a coating solution. The coating solution was gradually added to a Granurex GX20 (Freund, Japan) containing about 700 g of a sugar sphere at outlet temperature of about 40° C. to prepare granules.

### EXAMPLE 4

1.078 g of tamsulosin hydrochloride and 6.00 g of propylene glycol were added to 198 g of polyvinylacetate (Kollicoat SR30D, BASF Co.) and completely mixed to prepare a coating solution. The coating solution was gradually added to a Granurex GX20 (Freund, Japan) containing about 700 g of a sugar sphere at outlet temperature of about 40° C. to prepare granules.

### EXAMPLE 5

1.078 g of tamsulosin hydrochloride, 8.75 g of propylene glycol, 5 g of talc, and 5 g of titanium oxide were added to a combination of 250 g of Eudragit RS 30D (Degussa) and 41.667 g of Eudragit RL 30D (Degussa) and completely mixed to prepare a coating solution. The coating solution was gradually added to a Granurex GX20 (Freund, Japan) containing about 700 g of a sugar sphere at outlet temperature of about 40° C. to prepare primary granules.

2.58 g of propylene glycol, 0.87 g of povidone, 3.6 g of titanium oxide, and 3.6 g of talc were added to 86 g of polyvinylacetate (Kollicoat SR30D, BASF Co.) and completely mixed to prepare a coating solution. The coating solution was gradually added to the primary granules to prepare final granules.

### EXAMPLE 6

1.078 g of tamsulosin hydrochloride, 87.6 g of talc, and 13.14 g of propylene glycol were added to 438 g of Eudragit RS 30D (Degussa) and completely mixed to prepare a coating solution. The coating solution was gradually added to a Granurex GX20 (Freund, Japan) containing about 700 g of a sugar sphere at outlet temperature of about 40° C. to prepare primary granules.

4.95 g of propylene glycol, 1.669 g of povidone, and 19 g of talc were added to 165 g of polyvinylacetate (Kollicoat SR30D, BASF Co.) and completely mixed to prepare a coating solution. The coating solution was gradually added to the primary granules to prepare final granules.

### EXAMPLE 7

13.783 g of propylene glycol, 6.255 g of povidone, 25.1 g of talc, and 25.1 g of titanium dioxide were added to 454.86 g of Kollicoat SR30D (BASF Co.) and completely mixed to prepare a coating solution. The coating solution was gradually added to a Granurex GX20 (Freund, Japan) containing about 766 g of the primary granules prepared in Example 5 at outlet temperature of about 40° C. to prepare granules.

### EXAMPLE 8

11 g of propylene glycol was added to 369.24 g of Eudragit L100-55 (Degussa) and completely mixed to prepare a coating solution. The coating solution was gradually added to a Granurex GX20 (Freund, Japan) containing 1,000 g of the final granules prepared in Example 6 at outlet temperature of about 40° C. to prepare granules covered with enteric coating films.

### EXAMPLE 9

11 g of propylene glycol was added to 369.24 g of Eudragit L100-55 (Degussa) and completely mixed to prepare a coating solution. The coating solution was gradually added to a Granurex GX20 (Freund, Japan) containing about 823 g of the granules prepared in Example 2 at outlet temperature of about 40° C. to prepare granules covered with enteric coating films.

### COMPARATIVE EXAMPLE

1.078 g of tamsulosin hydrochloride and 15.764 g of propylene glycol were added to 525 g of Eudragit RL 30D (Degussa) and completely mixed to prepare a coating solution. The coating solution was gradually added to a Granurex GX20 (Freund, Japan) containing about 700 g of a sugar sphere at outlet temperature of about 40° C. to prepare granules.

### EXPERIMENTAL EXAMPLE

### Release Rate Test

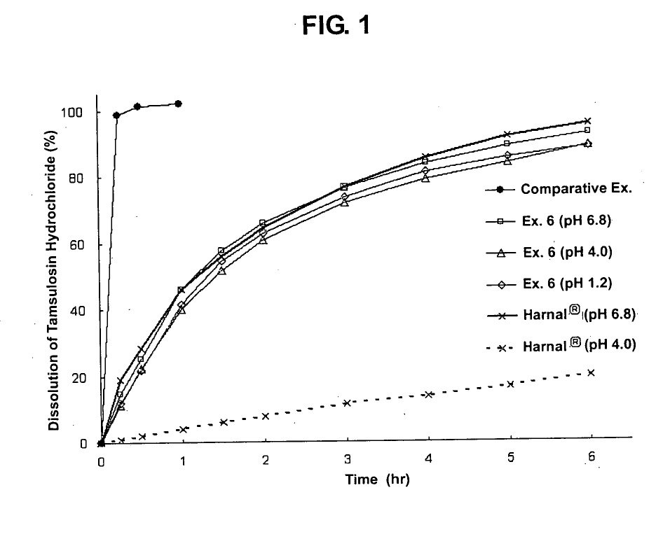

The release rates of the granules prepared in Example 6 and Comparative Example were measured in pH 1.2, 4.0, and 6.8 buffered solutions at a rate of 50 rpm by a paddle method. The release rate of a commercially available tamsulosin hydrochloride-containing formulation (Harnal, Yamanouchi Korea Co. Ltd.) was also measured. The results are shown in FIG. 1.

As seen from FIG. 1, the granules of the present invention efficiently controlled the drug release, as compared to those of Comparative Example. Also, they exhibited constant drug release property regardless of the ambient pH, unlike the commercially available tamsulosin hydrochloride-containing formulation.

1. A controlled-release formulation of tamsulosin hydrochloride, which comprises:
a granular core; and
a drug-coating layer coated on the granular core, comprising the tamsulosin hydrochloride and a release-controlling agent selected from the group consisting of
(a) a first copolymer of 1 part by weight of ethylacrylate, 2 parts by weight of methylmethacrylate, and 0.1 parts by weight of trimethylammonioethyl methacrylate chloride,
(b) a mixture of the first copolymer and a second copolymer of 1 part by weight of ethylacrylate, 2 parts by weight of methylmethacrylate, and 0.2 parts by weight of trimethylammonioethyl methacrylate chloride, the weight ratio between the first copolymer and the second copolymer being 1: 0.05 to 0.2, and
(c) polyvinylacetate.

2. The controlled-release formulation of claim 1, which further comprises a release-controlling layer coated on the drug-coating layer with the release-controlling agent.

3. The controlled-release formulation of claim 1, wherein a weight ratio between the tamsulosin hydrochloride and the release-controlling agent in the drug-coating layer is 1:10 to 1:200.

4. The controlled-release formulation of claim 2, wherein the release-controlling agent in the release-controlling layer is used in an amount of 20 to 80 parts by weight, based on 1 part by weight of the tamsulosin hydrochloride.

5. The controlled-release formulation of claim 1, wherein the drug-coating layer and/or the release-controlling layer further comprise(s) 0.1 to 5 parts by weight of a release-controlling adjuvant selected from the group consisting of polyvinylpyrrolidone, hydroxypropylmethyl cellulose, hydroxypropyl cellulose, and a mixture thereof, based on 1 part by weight of the tamsulosin hydrochloride.

6. The controlled-release formulation of claim 1, wherein the drug-coating layer and/or the release-controlling layer further comprise(s) a plasticizer selected from the group consisting of dibutylsebacate, triethylcitrate, propylene glycol, and a mixture thereof.

7. The controlled-release formulation of claim 1, which further comprises an enteric coating layer coated on the drug-coating layer or the release-controlling layer with an enteric coating agent selected from the group consisting of carboxymethylethyl cellulose, hydroxypropylmethyl cellulose phthalate, hydroxypropylmethyl cellulose acetate succinate, cellulose acetate phthalate, a copolymer of methacryic acid and ethylacrylate, a copolymer of methacrylic acid and methylmethacrylate, and shellac.

8. A process for preparing a controlled-release formulation of tamsulosin hydrochloride, which comprises:
preparing a drug-coating solution that comprises the tamsulosin hydrochloride and a release-controlling agent selected from the group consisting of (a) a first copolymer of 1 part by weight of ethylacrylate, 2 parts by weight of methylmethacrylate, and 0.1 parts by weight of trimethylammonioethyl methacrylate chloride, (b) a mixture of the first copolymer and a second copolymer of 1 part by weight of ethylacrylate, 2 parts by weight of methylmethacrylate, and 0.2 parts by weight of trimethylammonioethyl methacrylate chloride, the weight ratio between the first copolymer and the second copolymer being 1: 0.05 to 0.2, and (c) polyvinylacetate; and
spray-coating a granular core with the drug-coating solution to form a drug-coating layer.

9. The process of claim 8, which further comprises coating the drug-coating layer with the release-controlling agent to form a release-controlling layer.

10. The process of claim 8, wherein a weight ratio between the tamsulosin hydrochloride and the release-controlling agent in the drug-coating layer is 1:10 to 1:200.

11. The process of claim 9, wherein the release-controlling agent in the release-controlling layer is used in an amount of 20 to 80 parts by weight, based on 1 part by weight of the tamsulosin hydrochloride.

12. The process of claim 8, wherein the drug-coating layer and/or the release-controlling layer further comprise(s) 0.1 to 5 parts by weight of a release-controlling adjuvant selected from the group consisting of polyvinylpyrrolidone, hydroxypropylmethyl cellulose, hydroxypropyl cellulose, and a mixture thereof, based on 1 part by weight of the tamsulosin hydrochloride.

13. The process of claim 8, wherein the drug-coating layer and/or the release-controlling layer further comprise(s) a plasticizer selected from the group consisting of dibutylsebacate, triethylcitrate, propylene glycol, and a mixture thereof.

14. The process of claim 8, which further comprises coating the drug-coating layer or the release-controlling layer with an enteric coating agent selected from the group consisting of carboxymethylethyl cellulose, hydroxypropylmethyl cellulose phthalate, hydroxypropylmethyl cellulose acetate succinate, cellulose acetate phthalate, a copolymer of methacryic acid and ethylacrylate, a copolymer of methacrylic acid and methylmethacrylate, and shellac, to form an enteric coating layer.
